(19) United States
(12) Patent Application Publication
(10) Pub. No.: US 2019/0167534 A1
(43) Pub. Date: Jun. 6, 2019

(54) NOVEL COLOURING METHOD USING FILMS

(71) Applicant: Henkel AG & Co. KGaA, Duesseldorf (DE)

(72) Inventors: Konstantin Goutsis, Juechen (DE); Daniela Kessler-Becker, Leverkusen (DE)

(73) Assignee: Henkel AG & Co. KGaA, Duesseldorf (DE)

(21) Appl. No.: 16/209,079

(22) Filed: Dec. 4, 2018

(30) Foreign Application Priority Data
Dec. 4, 2017 (DE) 10 2017 221 826.6

(51) Int. Cl. A61K 8/02; A61K 8/365; A61K 8/34; A61K 8/49; A61Q 5/06

(52) U.S. Cl. CPC A61K 8/0204; A61K 8/365; A61K 8/345; A61K 2800/56; A61Q 5/065; A61K 2800/87; A61K 2800/432; A61K 8/4973

(57) ABSTRACT

The present disclosure relates to a method for colouring human hair, comprising the following steps:
(I) bringing a hair strand into contact with a colouring film, wherein the colouring film comprises at least one absorbent material and at least one substantive dye,
(II) applying a water-containing activator preparation to the hair strand and/or the colouring film,
(III) encasing the hair strand with the colouring film,
(IV) leaving the activator preparation to take effect on the hair strand and the colouring film for a period of from about 1 to about 60 minutes, and
(V) removing the colouring film.

The present disclosure also relates to a cosmetic product which makes it possible to colour hair in accordance with the above-described method.

### CROSS-REFERENCE TO RELATED APPLICATION

This application claims priority to German Patent Application No. 10 2017 221 826.6, filed Dec. 4, 2017, which is incorporated herein by reference in its entirety.

### TECHNICAL FIELD

The present application lies in the field of cosmetics and relates to a novel method for colouring hair with substantive dyes, which method makes it possible to produce streaking effects on hair.

### BACKGROUND

The changing of the form and colour of keratin fibres, in particular hair, represents a key area of modem cosmetics. The appearance of hair can hereby be adapted to current fashion trends and to the individual wishes of an individual person. For changing the colour of hair, a person skilled in the art knows a variety of colouring systems, depending on the requirements in regard to the colouration. Usually, oxidation dyes are used for permanent, intensive colourations having good fastness properties and good grey coverage. Such colouring agents usually comprise oxidation dye precursors, so-called developer components and coupler components, which create the actual dyes among each other under the influence of oxidising agents, such as hydrogen peroxide. Oxidation dyes are exemplified by excellent long-lasting colour results, but are also associated with a certain degree of hair damage. With the focus on colouring that results in a particularly low level of damage, the use of oxidation dyes therefore is not the method of first choice.

Hair colour can be changed temporarily by the use of substantive dyes. Here, dyes that have already fully formed diffuse from the colouring agent into the hair fibres. Compared to oxidative hair colouring, the colourations obtained with substantive dyes do not last as long and wash out more quickly. The grey coverage that can be achieved with substantive dyes is also generally worthy of improvement. The low level of hair damage caused by the colouring with substantive dyes, however, is advantageous.

For colouring with substantive dyes, various substance classes are available to a person skilled in the art, such as nitro dyes, anthraquinone dyes, azo dyes, or triarylmethane dyes. It is furthermore also possible to classify the dyes in various categories depending on their charge, Dyes with a cationic charge are generally referred to as “basic dyes”; dyes with anionic charge are known to a person skilled in the art by the term “acid dyes”.

A disadvantage of many substantive dyes is their often very pronounced staining of the skin. In particular, the group of acid dyes is known for very strongly staining the skin undesirably. During the colouring process, the colouring of the hair and the staining of skin occur in parallel. The more intense is the hair colouring, generally the stronger is also the staining of the skin of all body parts that come into contact during the colouring with the dye.

In order to give the hair a natural appearance, the “streaking effect” is very popular among users. Here, the colouring cream is applied merely to certain hair parts (streaks), such that a multi-tonal hair colour is produced. In the case of the streaking colouring technique, a liquid dye, which can be a cream, a gel or a solution, is usually applied by employing a brush or applicator. Here, the hair to be treated is generally selected with aluminium film or what is known as a “streaking cap”. This form of the streaking colouring effect known from the prior art is associated with various disadvantages.

On the one hand, the selective lightening of individual hair parts, such as bundles of hair fibres, strands of hair, and hairs on the crown of the head, can only be achieved with difficulty without suitable aids. Above all, a user wishing to colour the streaks himself must select individual strands of hair with an uncomfortable posture, in a time-consuming process, and with the aid of mirrors and applicators. This form of application is extremely uncomfortable and requires a lot of experience and skill.

On the other hand, there is the disadvantage of skin staining also in this streaking colouring technique, since here too a liquid or pasty colouring cream is used. When applying this liquid cream, it is practically impossible to prevent, besides the streaks themselves, all adjacent body parts (such as the scalp at the root of the hair, the skin in the area of the forehead, temples and neck) from also being wetted and stained by the dye.

### BRIEF SUMMARY

This disclosure provides a method for colouring human hair, comprising the following steps:
(I) bringing a hair strand into contact with a colouring film, wherein the colouring film comprises at least one absorbent material and at least one substantive dye,
(II) applying a water-containing activator preparation to the hair strand and/or the colouring film,
(III) encasing the hair strand with the colouring film,
(IV) leaving the activator preparation to take effect on the hair strand and the colouring film for a period of from about 1 to about 60 minutes, and
(V) removing the colouring film.

This disclosure also provides a cosmetic product, comprising (A) at least one colouring film comprising at least one absorbent material and at least one substantive dye, and (B) a water-containing activator preparation.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

One subject of the present disclosure is a cosmetic product which makes it possible to colour hair in accordance with the above-described method.

Another object of the present disclosure was therefore to provide a novel colouring method and a novel product which allow comfortable colouring by way of a streaking technique without staining the skin. Above all, the acid dyes, which are known for their extraordinarily high skin staining, should be able to be applied easily and also comfortably in this way. By way of the method and product, coloured strands with particularly good fastness properties (in particular good washing fastness).

It has now surprisingly been proven that the above problem can be solved outstandingly if a specific method is used to colour the hair, in which method a colouring film and an activator preparation are used.

Yet another subject of the present disclosure is a method for colouring human hair, comprising the following steps:
(I) bringing a hair strand into contact with a colouring film, wherein the colouring film comprises at least one absorbent material and at least one substantive dye,
(II) applying a water-containing activator preparation to the hair strand and/or the colouring film,
(III) encasing the hair strand with the colouring film,
(IV) leaving the activator preparation to take effect on the hair strand and the colouring film for a period of from about 1 to about 60 minutes, and
(V) removing the colouring film.

### Colouring Method by Way of the Streaking Technique

In the method as contemplated herein, the hair strands to be coloured are firstly brought into contact with a special colouring film. This colouring film comprises an absorbent or swellable material, on which at least one substantive dye is disposed.

Once the hair strand has been placed on or against the film, an aqueous activator preparation is applied to the hair strand and/or the colouring film. So that the colouring film then comes into full contact with the hair strand to be coloured, the hair strand is encased by the colouring film, i.e. the colouring film is wrapped, folded or wound around the hair strand. Due to the close contact between hair strand the colouring film, the activator preparation also acts both on the hair strand and on the film, and the absorbent material disposed on the colouring film absorbs the activator preparation. In this way, the substantive dye disposed on the film is mobilised and can diffuse to the hair strand. A reaction time of from about 1 to about 60 minutes is sufficient in order to colour the hair strand in this way, such that the colouring film can be removed thereafter.

With this method, the targeted and precise colouring of a hair strand is possible precisely at the point that is brought into direct contact both with the colouring film and with the activator composition.

(I) Bringing the Hair Strand into Contact with the Colouring Film

To start the method as contemplated herein, the hair strand that is to be coloured is brought into contact with a colouring film. For this purpose, the colouring film for example can be folded along its longitudinal axis and the strand placed in this fold. The hair strand brought into contact with the colouring film can be dry or already wetted with water.

Since in step (III) a liquid activator preparation is applied to the hair strand and/or the colouring film, the colouring film in step (I) is not fully folded or closed around the strand, and instead the hair strand is placed on the colouring film such that it is still possible to access the strand and the inner side of the film.

During the colouring, the part of the hair strand that is to be coloured is brought into contact with the colouring film. If a colouring of strands merely in the tip region is desired, preferably only the lower portion of the strand is brought into contact with the colouring film. If the strand is to be coloured over its entire length, it is advantageous to position the colouring film directly against the hairline and to place the entire strand on the colouring film.

One or more hair strands can be coloured using the method as contemplated herein. If a number of strands are to be coloured, each strand to be coloured is preferably encased by a separate colouring film, such that—depending on the number of desired coloured strands—a number of colouring films are used. Step (I) of the method as contemplated herein, i.e. the bringing of the strand to be coloured into contact with the colouring film, can thus firstly be repeated until all strands that are to be coloured have been placed on their corresponding colouring film. Step (II) of the method can then be performed.

When colouring a number of strands, steps (I) and (II) or also (I) to (III) of the method can also be repeated a number of times, such that ultimately all strands to be coloured are encased successively with the activator preparation and encased by a colouring film. In step (IV) the activator preparation is then left to take effect on all strands in parallel or at the same time.

### Absorbent Material

The colouring film is an application aid which is provided preferably in the form of a sheet or a strip. It is essential to the present disclosure here that the colouring film comprises at least one absorbent material. Here, the part of the film that comes into direct contact with the strand to be coloured is preferably made of an absorbent material.

An absorbent material as contemplated herein is understood to mean a material that can absorb liquid, in particular a water-containing or aqueous solution. An absorbent material as contemplated herein is synonymous with an absorptive or swellable material.

A first subject of the present disclosure is a method for colouring human hair, comprising the following steps:
(I) bringing a hair strand into contact with a colouring film, wherein the colouring film comprises at least one absorbent material and at least one substantive dye,
(II) applying a water-containing activator preparation to the hair strand and/or the colouring film,
(III) encasing the hair strand with the colouring film,
(IV) leaving the activator preparation to take effect on the hair strand and the colouring film for a period of from about 1 to about 60 minutes, and
(V) removing the colouring film.

A first subject of the present disclosure is a method for colouring human hair, comprising the following steps:
(I) bringing a hair strand into contact with a colouring film, wherein the colouring film comprises at least one absorbent material and at least one substantive dye,
(II) applying a water-containing activator preparation to the hair strand and/or the colouring film,
(III) encasing the hair strand with the colouring film,
(IV) leaving the activator preparation to take effect on the hair strand and the colouring film for a period of from about 1 to about 60 minutes, and
(V) removing the colouring film.

Materials based on cellulose, such as chromatography paper, filter paper, crepe paper, washi, China paper, or also woven or non-woven textile fibres are used as suitable absorbent or absorptive materials. The use of chromatography paper and filter paper is very particularly preferred.

Chromatography paper or also filter paper include refined pulps with a content of α-cellulose of more than about 95% and linters, i.e. short-fibre cotton, of high purity.

Washi is a hand-crafted, translucent paper from Japan, which can be obtained from various shrubs, such as the paper tree or the Japanese paper mulberry.

China paper is a particularly soft, absorptive, usually slightly yellowish paper. China paper can be produced for example from the bast paper mulberry, with admixture of ramie, cannabis, paddy straw, wheat straw, bamboo (poaceae) and/or rags

The aforementioned cellulose-based materials, in particular chromatography paper and filter paper, are able to absorb or soak up liquids, such as aqueous solutions. During this absorption, water molecules penetrate the non-crystalline regions of the material and in so doing spread apart the cellulose chains. The absorption capacity or absorbency of different cellulosic materials is very different here.

The method described hereinafter can be used as a measure for determining the absorption capability or also absorbency of a material, in particular one of the aforementioned cellulose-based papers (measurement of the absorption level):

A test strip of the absorbent material about 15 mm wide and about 250 mm long (for example chromatography paper) is suspended vertically via the narrow side in pre-filtered water (about 20° C.), such that the water rises through the capillaries of the strip. After a test time of about 30 minutes, the wetted part of the strip is measured in mm. The absorption level is specified in mm per about 30 min.

Very well-suited chromatography papers are obtainable for example from the company Sartorius Stedim Biotech SA. This company sells various types of chromatography paper, about 100% cotton linters, with a content of α-cellulose of more than about 98%.

| | | | Absorption level |
|---|---|---|---|
| Type | Weight (g/m²) | Thickness (mm) | (mm/30 min) |
| FN 1 | 90 | 0.19 | 145 |
| FN 2 | 125 | 0.25 | 145 |
| FN 3 | 90 | 0.19 | 95 |
| FN 4 | 125 | 0.24 | 95 |
| FN 5 | 90 | 0.18 | 60 |
| FN 6 | 125 | 0.22 | 60 |
| FN 7 | 150 | 0.32 | 145 |
| FN 75 | 200 | 0.41 | 145 |
| FN 8 | 280 | 0.55 | 170 |
| FN 30 | 320 | 0.90 | 240 |
| FN 100 | 195 | 0.35 | 115 |

Further suitable chromatography papers can be commercially procured for example from the company Whatman.

| | | | Absorption level |
|---|---|---|---|
| Type | Material | Thickness (mm) | (mm/30 min) |
| 3 MM CHR | Cellulose | 0.34 | 130 |
| 1 CHR | Cellulose | 0.18 | 130 |

Further suitable chromatography papers can also be commercially procured for example from the company Hahnemühl. The chromatography papers of this company include pure cotton linters with an alpha-cellulose content of >98%. These papers are available as sheets in the sizes 460×570 mm and 580×600 mm.

| | | | Absorption level |
|---|---|---|---|
| Type | Weight (g/m²) | Thickness (mm) | (mm/30 min) |
| 2043a | 90 | 0.17 | 105 |
| 2043b | 120 | 0.22 | 105 |
| 2668 | 320 | 0.90 | 250 |

As contemplated herein, an absorbent material or absorptive material is a material that has an absorption level (specified in mm/30 min) of at least about 20, preferably at least about 30, more preferably at least about 55, wherein the absorption level was measured in accordance with the above-described method.

Within the scope of a further embodiment, a particularly preferred method for colouring human hair comprises the step of
(I) bringing a hair strand into contact with a colouring film, wherein the colouring film comprises at least one absorbent material and at least one substantive dye,

A cellulose-based material is understood to be a material with a cellulose content—in relation to the total content of the absorbent or absorptive material—of at least about 80% by weight, preferably of at least about 90% by weight, and particularly preferably of at least about 95% by weight.

Within the scope of a further embodiment, a particularly preferred method for colouring human hair comprises the step of
(I) bringing a hair strand into contact with a colouring film, wherein the colouring film comprises at least one absorbent or absorptive material selected from the group of chromatography paper, filter paper, crepe paper, washi, China paper, and/or woven or non-woven cellulose fibres.

### Single- or Multi-Layer Film

The colouring film can comprise one or more plies or layers. If the colouring film for example includes just one layer, it can be manufactured in the form of a sheet or strip from one of the aforementioned preferred absorbent materials. Within the scope of this embodiment, the hair strand to be coloured for example can be brought into contact with a sheet or strip made of chromatography paper (with at least one substantive dye disposed thereon).

A disadvantage of absorbent or absorptive materials, however, lies in the fact that their stability decreases as soon as they come into contact with liquids. In the moist state, the papers used as colouring film can then quickly tear or rip. Although the colouring is possible with this single-layer film, it can be perceived by the user to be uncomfortable.

For this reason, it is preferred if a multi-layer film is used as colouring film which, besides a first layer made of absorbent material, also comprises at least one further layer, wherein this further layer is used as a stabilisation and/or protective layer.

The first layer made of absorbent material in this content constitutes the (inner) layer, which is in direct contact with the hair strand to be coloured. The second (outer) layer is then the stabilisation or protective layer, which for example can be a film made of metal or a polymeric material.

Within the scope of a further embodiment, a particularly preferred method for colouring human hair comprises the step of
(I) bringing a hair strand into contact with a multi-layer colouring film, wherein the inner layer of the film, which is brought into contact with the hair strand, includes an absorbent material.

Within the scope of a further embodiment, a very particularly preferred method for colouring human hair comprises the step of
(I) bringing a hair strand into contact with a multi-layer colouring film, wherein the inner layer of the film, which is brought into contact with the hair strand, includes an absorbent material
selected from the group of chromatography paper, filter paper, washi, China paper and/or woven or non-woven cellulose fibres.

The colouring film is particularly preferably provided as a multi-layer film which comprises at least two plies or layers. The inner layer, which is brought directly into contact with the hair strand to be coloured, preferably comprises an absorbent, absorptive or swellable material. In other words, the layer which comes into direct contact with the strand to be coloured is produced from an absorbent, absorptive or swellable material.

The outer layer of the film does not necessarily come into direct contact with the hair strand to be coloured and includes a layer of metal or a polymeric material.

Within the scope of one embodiment, a particularly preferred method for colouring human hair comprises the step of
(I) bringing a hair strand into contact with a multi-layer colouring film, wherein the outer layer of the film includes a layer made of metal or a polymeric material.

Within the scope of this embodiment, the outer layer of the film can be produced for example from aluminium, iron, iron oxides, tin, zinc and/or mixtures thereof.

What is very particularly preferred is the
(I) bringing of a hair strand into contact with a multi-layer colouring film, wherein the outer layer of the film comprises a layer made of aluminium, iron, iron oxides, tin and/or zinc, very particularly preferably aluminium.

Within the scope of a further embodiment, the outer layer of the film can be produced from a polymeric material. This polymeric material is an organic polymeric material as contemplated herein. This material for example can be polypropylene, polyethylene, polyester, polyamide or polyvinyl alcohol, and/or mixtures of these polymers.

Within the scope of this embodiment, a preferred method for colouring human hair comprises the step of
(I) bringing a hair strand into contact with a multi-layer colouring film, wherein the outer layer of the film comprises a layer made of polypropylene, polyethylene, polyester, polyamide or polyvinyl alcohol, and/or mixtures of these polymers.

### Substantive Dyes

As a further feature essential to the present disclosure, the colouring film as contemplated herein comprises a substantive dye. This substantive dye is preferably present in finely dispersed and in solid form on the film and is preferably applied directly to, or in the direct vicinity of the absorbent material.

A very particularly well-suited colouring film can be produced for example in the following way:

A solution or suspension concentrated to the greatest possible extent is firstly produced from a substantive dye in water. To improve the solubility, a solvent and/or an acid can be added to this solution. An absorbent material (which for example can be chromatography paper, filter paper, crepe paper, etc.) provided in sheet form is then dipped into this solution, such that the absorbent material is fully saturated with the dissolved/suspended dye. The absorbent material is then dried. In order to accelerate the drying process, a war air fan or a hair-dryer can be used, for example. This absorbent material penetrated by the substantive dye then represents the inner layer of the colouring film. In order to increase the stability and for protection, the absorbent material is now also adhered to a further film layer, such as an aluminium film or a polyethylene film. The aluminium film or the PE film constitutes the outer layer of the now multi-layer colouring film. The colouring film produced in this way is applied such that the absorbent material is located “inwardly”, i.e. in direct contact with the hair strand.

If the above-described method is applied, the absorbent material of the colouring film is coloured by the substantive dye, or in other words is penetrated by the dye.

Within the scope of a further embodiment, a particularly preferred method for colouring human hair comprises the step of
(I) bringing a hair strand into contact with a colouring film, wherein the absorbent material is coloured with at least one substantive dye.

Within the scope of a further embodiment, a particularly preferred method for colouring human hair comprises the step of
(I) bringing a hair strand into contact with a colouring film, wherein the absorbent material is penetrated by at least one substantive dye.

In principle, all dyes that are usually used in hair colouring can be used as substantive dyes.

Suitable cationic substantive dyes are cationic triphenylmethane dyes, such as Basic Blue 7, Basic Blue 26, Basic Violet 2 and Basic Violet 14, aromatic systems which are substituted with a quaternary nitrogen group, such as Basic Yellow 57, Basic Red 76, Basic Blue 99, Basic Brown 16 and Basic Brown 17, cationic anthraquinone dyes, such as HC Blue 16 (Bluequat B) and substantive dyes which contain a heterocycle comprising at least one quaternary nitrogen atom, in particular Basic Yellow 87, Basic Orange 31 and Basic Red 51. The cationic substantive dyes that are sold under the trade name Arianor are likewise suitable cationic substantive dyes as contemplated herein.

Suitable non-ionic substantive dyes are in particular non-ionic nitro and quinone dyes and neutral azo dyes. Preferred non-ionic substantive dyes are the compounds known under the international names or trade names HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4 and Disperse Black 9, and 1,4-diamino-2-nitrobenzene, 2-amino-4-nitrophenol, 1,4-bis (2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

Within the group of substantive dyes, acid dyes have very good fastness properties, but also belong to the classes of dyes having the most severe skin staining. If acid dyes are used in the method as contemplated herein, the skin staining can be particularly effectively avoided in this way, and the method is therefore very particularly advantageous for the user.

Substantive dyes can be divided on the basis of the charge into cationic dyes (also referred to alternatively as basic dyes), non-ionic dyes, and anionic dyes (also referred to as acid dyes). Acid dyes are understood to be substantive dyes that have at least one carboxylic acid grouping (—COOH) and/or a sulfonic acid grouping (—SO₃H). Depending on the pH value, the protonated forms (—COOH, —SO₃H) of the carboxylic acid or sulfonic acid groupings are present in equilibrium with their deprotonated forms (—COO⁻, —SO₃⁻). With decreasing pH value, the proportion of the protonated forms rises. If substantive dyes are used in the form of their salts, the carboxylic acid groups or sulfonic acid groups are present in deprotonated form and are neutralised in order to maintain the electroneutrality with corresponding stoichiometric equivalent of cations (for example Na cation or K cations).

Suitable acid dyes can be selected for example from one or more compounds from the following group: Acid Yellow 1 (D&C Yellow 7, Citronin A, Ext. D&C Yellow No. 7, Japan Yellow 403 CI 10316, COLIPA n^{o} B001), Acid Yellow 3 (COLIPA n^{o}: C 54, D&C Yellow N^{o} 10, Quinoline Yellow, E104, Food Yellow 13), Acid Yellow 9 (CI 13015), Acid Yellow 17 (CI 18965), Acid Yellow 23 (COLIPA n^{o} C 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n^{o} C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I.14720), Acid Red 18 (E124, Red 18; CI 16255), Acid Red 27 (E 123, CI 16185, C-Rot 46, Echtrot D, FD&C Red Nr. 2, Food Red 9, Naphtholrot S), Acid Red 33 (Red 33, Fuchsia Red, D&C Red 33, CI 17200), Acid Red 35 (CI C.I.18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n^{o} 106 Pontacyl Brilliant Pink), Acid Red 73 (CI 27290), Acid Red 87 (Eosin, CI 45380), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet n^{o} 2, C.I. 60730, COLIPA n^{o} C063), Acid Violet 49 (CI 42640), Acid Violet 50 (CI 50325), Acid Blue 1 (Patent Blue, CI 42045), Acid Blue 3 (Patent Blau V, CI 42051), Acid Blue 7 (CI 42080), Acid Blue 104 (CI 42735), Acid Blue 9 (E 133, Patentblau AE, Amidoblau AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brillantsäuregrün BS, C.I. 44090, Acid Brilliant Green BS, E 142), Acid Black 1 (Black n^{o} 401, Naphthalene Black 10B, Amido Black 10B, CI 20 470, COLIPA n^{o} B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

Within the scope of a further embodiment, a particularly preferred method for colouring human hair comprises the step of
(I) bringing a hair strand into contact with a colouring film, wherein the colouring film comprises at least one absorbent material and at least one anionic substantive dye, which is preferably selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 18, Acid Red 27, Acid Red 33, Acid Red 35, Acid Red 51, Acid Red 52, Acid Red 73, Acid Red 87, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 7, D&C Yellow 8, D&C Orange 4, D&C Green 5, D&C Orange 10, D&C Orange 11, D&C Red 21, D&C Red 27, D&C Red 33, D&C Violet 2 and/or D&C Brown 1.

Within the scope of a further embodiment, a particularly preferred method for colouring human hair comprises the step of
(I) bringing a hair strand into contact with a colouring film, wherein the colouring film comprises at least one absorbent material, which is penetrated by at least one anionic substantive dye from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 18, Acid Red 27, Acid Red 33, Acid Red 35, Acid Red 51, Acid Red 52, Acid Red 73, Acid Red 87, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 7, D&C Yellow 8, D&C Orange 4, D&C Green 5, D&C Orange 10, D&C Orange 11, D&C Red 21, D&C Red 27, D&C Red 33, D&C Violet 2 and/or D&C Brown 1.

The substantive dye(s) can be applied in different amount ranges to the absorbent material. The amount ranges that the absorbent material can absorb are dependent here, amongst other things, on the dimensions and the thickness of the absorbent material. For example, suitable usage amounts are from about 0.0001 to about 0.1 g dye per g absorbent material. Preferred usage amounts are from about 0.001 to about 0.1 g dye per g absorbent material.

### Acids on the Absorbent Material

To further accelerate the colouring process, the colouring film as contemplated herein can additionally also comprise at least one acid. The acid—precisely the substantive dye—is preferably present in finely dispersed and in solid form on the film and is preferably applied directly to, or in the direct vicinity of the absorbent material.

Suitable acids here are for example inorganic acids, such as hydrochloric acid, sulfuric acid and/or phosphoric acid. However, organic acids, such as acetic acid, lactic acid, citric acid, tartaric acid, malic acid, 1-hydroxyethane-1,1-diphosphonic acid, 2,6-dipicolinic acid, benzoic acid maleic acid, succinic acid, oxalic acid, ascorbic acid, phytic acid and/or gluconic acid, can also likewise be used. In this context the odourless organic acids that are solid at room temperature are preferred, such as citric acid, tartaric acid and/or malic acid.

A corresponding film as contemplated herein can be produced for example in the following way:

A solution or suspension concentrated to the greatest possible extent is firstly produced from a substantive dye in water. An acid (for example citric acid) is added to this solution. A chromatography paper is then dipped into this solution, such that the paper fully soaks up the solution (which contains dye and acid). The chromatography paper is then dried at room temperature or with the aid of a hair-dryer. This chromatography paper now penetrated by a substantive dye and the acid the constitutes the inner layer of the colouring film. In order to increase the stability and for protection, the chromatography paper is glued to an aluminium film. The aluminium film constitutes the outer layer of the now multi-layer colouring film.

Within the scope of a further embodiment, a particularly preferred method for colouring human hair comprises the step of
(I) bringing a hair strand into contact with a colouring film, wherein the colouring film comprises at least one absorbent material and at least one substantive dye and at least one acid.

If the above-described method is applied, the absorbent material of the colouring film is penetrated by the substantive dye and by the acid.

Within the scope of a further embodiment, a particularly preferred method for colouring human hair comprises the step of
(I) bringing a hair strand into contact with a colouring film, wherein the colouring film comprises at least one absorbent material which is penetrated by at least one substantive dye and by at least one acid.

The substantive acid(s) can be applied in different amount ranges to the absorbent material. The amount ranges that the absorbent material can absorb are dependent here, amongst other things, on the dimensions and the thickness of the absorbent material. For example, suitable usage amounts are from about 0.0001 to about 0.1 g acid per g absorbent material. Preferred usage amounts are from about 0.001 to about 0.1 g acid per g absorbent material.

Step (II) of the Method: Applying a Water-Containing Activator Preparation to the Hair Strand and/or the Colouring Film,

In step (II) of the method as contemplated herein, a water-containing activator preparation is applied to the hair strand and/or the colouring film.

Within the scope of one embodiment, once the hair strand to be coloured has been brought into contact with the colouring film in step (I) of the method as contemplated herein, a water-containing activator preparation is then applied to the hair strand and/or the colouring film (II).

Within the scope of the method as contemplated herein, step (II) however does not necessarily have to be performed after step (I). In a further embodiment it is therefore also possible to first apply the water-containing activator preparation to the hair strand and/or the colouring film (II) and then to bring the hair strand into contact with the colouring film.

The present disclosure therefore comprises a method for colouring human hair, comprising the following steps in the specified order
(I) bringing a hair strand into contact with a colouring film, wherein the colouring film comprises at least one absorbent material and at least one substantive dye,
(II) applying a water-containing activator preparation to the hair strand and/or the colouring film,
(III) encasing the hair strand with the colouring film,
(IV) leaving the activator preparation to take effect on the hair strand and the colouring film for a period of from about 1 to about 60 minutes, and
(V) removing the colouring film.

The present disclosure therefore likewise also comprises a method for colouring human hair, comprising the following steps in the specified order
(I) applying a water-containing activator preparation to a hair strand and/or a colouring film, wherein the colouring film comprises at least one absorbent material and at least one substantive dye,
(I) bringing the hair strand into contact with the colouring film
(III) encasing the hair strand with the colouring film,
(IV) leaving the activator preparation to take effect on the hair strand and the colouring film for a period of from about 1 to about 60 minutes, and
(V) removing the colouring film.

Whether, when colouring a hair strand, step (I) or step (II) is performed first can be dependent for example on the personal preference of the user and the position of the hair strand to be coloured.

The colouring of defined strands—without the undesirable colouring also of adjacent hair parts—is better possible, however, if step (I) is performed first for each strand, followed then by step (II).

The hair strand to be coloured is wetted by the application of the water-containing activator preparation. If the activator preparation contains an acid, the hair strand is also acidified at the same time. The colouring film—either directly by the application of the activator preparation or indirectly by the contact with the wetted hair strand—is likewise wetted with the water-containing activator preparation, which causes the absorbent material to be fully saturated with the activator preparation. This causes the substantive dye previously present in solid form to dissolve (or be suspended) and to then be mobilised (the dye is “activated”). Here, the substantive dye(s) is/are mobilised precisely at the point of the film that was previously wetted with the activator preparation. Accordingly, only the point of the strand that is in contact with the colouring film at this point is also coloured.

During this process, the absorbent material absorbs the liquid activator preparation. This absorption on the one hand enables the dissolution of the substantive dye, but furthermore additionally also prevents the liquid activator preparation from dripping down or running down. In this way, the colouring with streaking effect can be performed very particularly precisely.

When applying the water-containing activator preparation, this can be applied either only to the hair strand or can be applied only to the colouring film. An application of the activator preparation both to the hair strand and to the colouring film is also possible. Since, after step (II), the complete encasement of the hair strand with the colouring film (III) follows, the direct contact between hair strand and absorbent material of the colouring film is ensured in all of these variants by step (III). In any case, the dissolved or mobilised substantive dye can thus diffuse towards the hair strand and colour it.

The activator preparation is water-containing or aqueous and can be formulated for example as a cream, emulsion, a gel, a solution, or a spray. This preparation can be applied accordingly by employing a brush, an applicator, or a comb. The spraying of the activator preparation or the application by (gloved) hands is also possible.

The activator preparation is used to dissolve or suspend the substantive dye and in this way to enable the transfer thereof from the absorbent material to the hair strand. For this purpose, the activator preparation is aqueous or contains water. In order to ensure a particularly good and quick dissolution of the substantive dye, the activator preparation can preferably additionally contain at least one solvent.

Suitable solvents are benzyl alcohol, phenoxyethanol, ethylene carbonate, propylene carbonate, butylene carbonate, glyceryl carbonate, ethanol, ethylene glycol, isopropanol, 1,2-propylene glycol, 1,3-propylene glycol, glycerol, n-butanol and 1,3-butylene glycol.

Benzyl alcohol is an aromatic alcohol of formula (I), and 2-phenoxyethan-1-ol is an aromatic alcohol of formula (II).

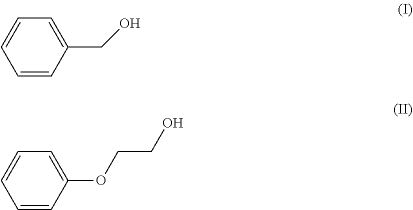 [SMILES: OCc1ccccc1 ; OCCOc1ccccc1]

The solvent propylene carbonate, when used as a solvent, likewise demonstrates very good suitability. Propylene carbonate is alternatively also referred to as 4-methyl-1,3-dioxolan-2-one and has the structure of formula (III).

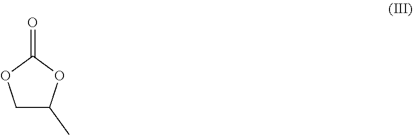

Benzyl alcohol, phenoxyethanol and propylene carbonate are very particularly well suited for rapid dissolution of the substantive dyes. Particularly in the case of anionic dyes, these solvents have an excellent dissolution capability.

Within the scope of a further very particularly preferred embodiment, a method as contemplated herein is exemplified in that the activator preparation contains at least one solvent selected from the group of benzyl alcohol, phenoxyethanol, propylene carbonate, ethylene carbonate, 1,2-propylene glycol, butylene carbonate, glyceryl carbonate, ethanol, ethylene glycol, isopropanol, 1,3-propylene glycol, glycerol, n-butanol and 1,3-butylene glycol, particularly preferably selected from the group of benzyl alcohol, phenoxyethanol and propylene carbonate.

The activator preparation preferably contains one or more solvents in a total amount of from about 1.0 to about 15.0% by weight, preferably from about 4.0 to about 13.5% by weight, more preferably from about 7.0 to about 13.0% by weight, and particularly preferably from about 8.0 to about 12.0% by weight, in relation to the total weight of the activator preparation.

The solubility of the substantive dyes can be further optimised by varying the pH level. Within the scope of a further embodiment it is therefore particularly preferred if the activator preparation also contains at least one acid. Above all if the absorbent material comprises one or more acid dyes (or is penetrated by one or more acid dyes), the dissolution or the transfer of the dye to the to the hair strand can be optimised further still by application of an acidic activator preparation.

Particularly well-suited acids can be selected in this regard from the acids that are selected from the group of lactic acid, citric acid, tartaric acid, malic acid, 1-hydroxyethane-1,1-diphosphonic acid, 2,6-dipicolinic acid, benzoic acid, phosphoric acid, sulfuric acid, hydrochloric acid, maleic acid, succinic acid, oxalic acid, ascorbic acid, phytic acid and/or gluconic acid.

Within the scope of a further very particularly preferred embodiment a method as contemplated herein is exemplified in that the activator preparation contains at least one acid selected from the group of lactic acid, citric acid, tartaric acid, malic acid, 1-hydroxyethane-1,1-diphosphonic acid, 2,6-dipicolinic acid, benzoic acid, phosphoric acid, sulfuric acid, hydrochloric acid, maleic acid, succinic acid, oxalic acid, ascorbic acid, phytic acid and/or gluconic acid.

Anionic dyes have the best colouring effect in the acidic pH range. For this reasons, it is particularly advantageous to add amounts of acids to the activator preparation such that the pH value of said preparation is acidic. Particularly intense washable colourings were obtained if the activator preparation was set to a pH value in the range of from about 1.0 to about 7.0, preferably from about 1.3 to about 6.0, more preferably from about 1.3 to about 5.0, even more preferably from about 1.8 to about 4.0, and very particularly preferably from about 2.0 to about 3.5.

Within the scope of a further very particularly preferred embodiment a method as contemplated herein is exemplified in that the activator preparation has a pH value of from about 1.0 to about 7.0, preferably from about 1.3 to about 6.0, more preferably from about 1.3 to about 5.0, even more preferably from about 1.8 to about 4.0, and very particularly preferably from about 2.0 to about 3.5.

The activator preparation can also contain additional active substances, auxiliaries and additives in order to set further desired properties of the product.

The activator preparation as contemplated herein is preferably provided as a liquid preparation, and the activator preparation is therefore optionally mixed additionally with a further surface-active substance, wherein surface-active substances are referred to as surfactants or as emulsifiers depending on the field of application: They are preferably selected from anionic, zwitterionic, amphoteric and non-ionic surfactants and emulsifiers.

Activator preparations suitable as contemplated herein are exemplified in that the product additionally contains at least one anionic surfactant. Preferred anionic surfactants are fatty acids, alkyl sulfates, alkyl ether sulfates, and ether carboxylic acids with from about 10 to about 20 C atoms in the alkyl group and up to about 16 glycol ether groups in the molecule.

Activator preparations suitable as contemplated herein are exemplified in that the product additionally contains at least one zwitterionic surfactant. Preferred zwitterionic surfactants are betaines, N-alkyl-N,N-dimethylammonium glycinates, N-acyl-aminopropyl-N,N-dimethylammonium glycinates, and 2-alkyl-3-carboxymethyl-3-hydroxyethyl-imidazolines. A preferred zwitterionic surfactant is known under the INCI name Cocamidopropyl Betaine.

Activator preparations suitable as contemplated herein are exemplified in that the product additionally contains at least one amphoteric surfactant. Preferred amphoteric surfactants are N-alkyl glycines, N-alkyl propionic acids, N-alkylaminobutyric acids, N-alkyliminodipropionic acids, N-hydroxyethyl-N-alkylamidopropylglycines, N-alkyl taurines, N-alkyl sarcosines, 2-alkylaminopropionic acids and alkylaminoacetic acids. Particularly preferred amphoteric surfactants are N-coco-alkylaminopropionate, coco-acylaminoethylaminopropionate and C₁₂-C₁₈ acyl sarcosine.

It has also proven advantageous if the activator preparation contains further non-ionogenic surface-active substances. Preferred non-ionic surfactants have proven to be alkylpolyglycosides and alkylene oxide addition products with fatty alcohols and fatty acids, with in each case from about 2 to about 30 mol ethylene oxide per mol fatty alcohol or fatty acid. Preparations having excellent properties are also obtained if they contain fatty acid esters of ethoxylated glycerol as non-ionic surfactants.

The non-ionic, zwitterionic or amphoteric surfactants are used in amounts of from about 0.1 to about 45% by weight, preferably from about 1 to about 30% by weight, and very particularly preferably from about 1 to about 15% by weight, in relation to the total amount of the product.

The activator preparations as contemplated herein can also contain at least one thickener. There are in principle no limitations in respect of this thickener. Both organic and purely inorganic thickeners can be used.

Suitable thickeners are anionic, synthetic polymers, cationic, synthetic polymers, naturally occurring thickeners, such as non-ionic guar gums, scleroglucan gums, or xanthan gums, gum arabic, gum ghatti, karaya gum, tragacanth gum, carrageenan gum, agar-agar, locust bean flour, pectins, alginates, starch fractions, and derivatives such as amylose, amylopectin, and dextrins, as well as cellulose derivatives such as methylcellulose, carboxyalkylcelluloses, and hydroxyalkylcelluloses, non-ionic, fully synthetic polymers such polyvinyl alcohol or polyvinylpyrrolidinone, and inorganic thickeners, in particular sheet silicates such as bentonite, in particular smectites, such as montmorillonite or hectorite.

The activator preparations as contemplated herein can also contain further active substances, auxiliaries and additives, such as non-ionic polymers, such as vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or non-volatile, straight-chain, branched or cyclic, crosslinked or noncrosslinked polyalkylsiloxanes (such as dimethicones or cyclomethicones), polyarylsiloxanes, and/or polyalkylarylsiloxanes, particularly polysiloxanes with organofunctional groups, such as substituted or unsubstituted amines (amodimethicones), carboxyl, alkoxy, and/or hydroxyl groups (dimethicone copolyols), linear polysiloxane(A)-polyoxyalkylene(B) block copolymers, grafted silicone polymers; cationic polymers such as quaternised cellulose ethers, polysiloxanes with quaternary groups, dimethyldiallylammonium chloride polymers, acrylamide-dimethyldiallylammonium chloride copolymers, dimethylaminoethylmethacrylate-vinylpyrrolidinone copolymers quaternised with diethylsulfate, vinylpyrrolidinone-imidazolinium-methochloride copolymers, and quaternised polyvinyl alcohol; zwitterionic and amphoteric polymers; anionic polymers such as polyacrylic acids or crosslinked polyacrylic acids; structurants such as glucose, maleic acid, and lactic acid, hair-conditioning compounds such as phospholipids, for example, lecithin and kephalins; perfume oils, dimethyl isosorbide, and cyclodextrins; dyes for staining the product; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; vegetable oils; light stabilisers and UV blockers; active substances such as panthenol, pantothenic acid, pantolactone, allantoin, pyrrolidinone carboxylic acids, and salts thereof, as well as bisabolol; polyphenols, particularly hydroxy cinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetration agents such as propylene glycol monoethyl ether, carbonates, hydrogen carbonates, guanidines, ureas, and primary, secondary, and tertiary phosphates; opacifiers such as latex, styrene/PVP, and styrene/acrylamide copolymers; pearlescent agents such as ethylene glycol mono- and distearate and PEG-3 distearate; pigments and propellants such as propane-butane mixtures, N₂O, dimethyl ether, CO₂, and air.

The selection of these further substances is made by a person skilled in the art in accordance with the desired properties of the activator preparation. With regard to further optional components and the used amounts of these components, reference is made expressly to the relevant handbooks known to a person skilled in the art. The additional active substances and auxiliaries are used in the products as contemplated herein preferably in amounts of, in each case, from about 0.0001 to about 25% by weight, in particular from about 0.0005 to about 15% by weight, in relation to the total weight of the activator preparation.

Step (III) of the Method: Encasing the Hair Strand with the Colouring Film,

In order to ensure the uniform colouring of the hair strand, the hair strand (or all parts of a hair strand that are to be coloured) is now encased fully with the colouring film in step (III) of the method. The encasing of the hair strand with the colouring film is understood for example to mean a folding over, covering or wrapping.

Since the full encasing of the hair strand is expediently performed only following the application of the activator preparation (II), step (III) of the method is performed as contemplated herein after steps (I) and (II).

At this point of the method as contemplated herein, the hair strand for example may now lie in the fold of the colouring film folded parallel to its longitudinal axis. In order to encase the hair strand with the colouring film, the colouring film for example can now be folded together and optionally folded again. In this way, the hair strand is surrounded on all sides by the film (or by the absorbent material of the film). It is essential during the encasing process that each hair strand is in direct contact with the absorbent material of the colouring film at all points at which said hair strand is to be to be coloured.

To improve the contact, the strand covered by the colouring film (or encased by the colouring film) can now be lightly massaged or squeezed using the hands.

### Step (IV) of the Method: Leaving the Activator Preparation to Take Effect on the Hair Strand and the Colouring Film,

Once the hair strand has been encased by the colouring film, the colouring process occurs. Due to the close contact between hair strand and colouring film, the activator preparation now acts both on the hair strand and on the colouring film (or on the absorbent material of the colouring film). Here, the substantive dye dissolves and diffuses into the hair strand.

The colouring period can be selected by the user depending on the desired colour intensity. An initial colouring result can be seen after just one minute. If very intense colouring is desired, the user (or hairdresser) can also leave the activator preparation to take effect on the hair strand and colouring film for up to about 60 minutes.

A particularly well-suited method comprises the step of
(IV) leaving the activator preparation to take effect on the hair strand and the colouring film for a period of time of from about 1 to about 60 minutes, preferably from about 3 to about 50 minutes, more preferably from about 5 to about 45 minutes, and particularly preferably from about 10 to about 40 minutes.

During the reaction time of the activator preparation, it can be advantageous to assist the colouring process by the addition of heat. The heat can be supplied by an external heat source, such as warm air of a warm air fan, and by the body temperature of the user. In the case of the latter possibility, the entire head for example can also be covered by a hood.

### Step (V) of the Method: Removing the Colouring Film.

Once the colouring process is complete—i.e. following step (IV)—the colouring film is removed. If a number of strands have been coloured, all colouring films are at this point removed or the hair strands are unwrapped again from the films. The hair can then be washed with water—optionally with use of a shampoo or conditioner.

A further optional step is the drying of the hair.

### Cosmetic Product

In order to apply the method as contemplated herein it is particularly comfortable for the user if all necessary constituents are provided to him in the form of a cosmetic product.

A second subject of the present disclosure is therefore a cosmetic product, comprising

(A) at least one colouring film as has already been disclosed in detail in the description of the first subject of the present disclosure, and
(B) a water-containing activator preparation as has already been disclosed in detail in the description of the first subject of the present disclosure, and

In other words, a second subject of the present disclosure is therefore a cosmetic product, comprising

(A) at least one colouring film, wherein the colouring film comprises at least one absorbent material and at least one substantive dye, and
(B) a water-containing activator preparation, which preferably contains at least one solvent and/or at least one acid.

The design of the at least one colouring film has already been detailed in the description of the method as contemplated herein.

The design of the activator preparation has likewise been detailed in the description of the method as contemplated herein.

Should the applicant wish to colour just on strand, the provision of one colouring film is sufficient.

Generally, however, colouring with a streaking effect involves the colouring of a number of strands. Since a separate colouring film is necessary for the colouring of each strand, it is particularly preferred if the cosmetic product as contemplated herein comprises a set of colouring films—for example about 5, about 10, about 15 or also about 20 colouring films.

The colouring films (A) preferably have a sheet-like or also strip-like form; thus, they can be provided packaged in a stack in a box. It is also possible to offer the colouring films in the form of a roll, wherein the individual sheets or strips are separated from one another by perforations.

A particularly preferred cosmetic product therefore comprises

(A) a set of colouring films, wherein each colouring film has been disclosed in detail in the description of the first subject of the present disclosure, and
(B) a water-containing activator preparation as has already been disclosed in detail in the description of the first subject of the present disclosure.

The water-containing activator preparation, besides water, preferably also contains solvents and/or acids, and optionally also surfactants and/or emulsifiers and/or thickeners. The activator preparation can thus be present for example packaged in the form of a gel, an emulsion, a solution or a spray. For the packaging of the corresponding formulations, a person skilled in the art will choose the best-suitable vessel, which for example can a container, a bottle, a tube, a foam container, a pump foam container, or a sachet.

If the product is to enable multiple colouring processes with streaking effect, the water-containing activator preparation can also be present packaged in a number of portions or containers. These can be, for example, a number of small bottles or sachets.

With regard to the preferred embodiments of the cosmetic product as contemplated herein, that which has been said with regard to the method as contemplated herein applies mutatis mutandis.

### EXAMPLES

1. Production of the Colouring Film

The following colouring solution was firstly produced:

| Colouring solution (FL) | FL1 | FL2 |
|---|---|---|
| Acid Red 33 | 1.0 | 1.0 |
| Citric acid | — | 4.0 |
| Water | to 100 | to 100 |

The following chromatography paper was used:

Hahnemühle from pure cotton linters with an alpha-cellulose content of >98%.

| | | | Absorption level |
|---|---|---|---|
| Type | Weight (g/m²) | Thickness (mm) | (mm/30 min) |
| 2043b | 120 | 0.22 | 105 |

Sheets measuring 5 cm wide and 25 cm long were cut from the chromatography paper. The chromatography paper was then drizzled with one of the previously produced colouring solutions (FL1 and FL2) (1.2 g colouring solution were used in each case per g of paper). The strips of the chromatography paper coloured in this way were then blow-dried.

After the drying, the chromatography paper was then adhesively bonded to an aluminium film) dimensions 6 cm×27 cm).

2. Production of the Activator Preparation

The following activator preparation was firstly produced:

| Activator preparation (AZ) | AZ1 |
|---|---|
| Lactic acid (80% in water) | 0.5 |
| Benzyl alcohol | 5.0 |
| 1,2-propanediol | 5.0 |
| Xanthan | 1.0 |
| Water (dist.) | to 100 |

3. Colouring

Hair strands (Kerling 9-0) were measured colorimetrically using a colorimeter from the company Datacolor, model Spectraflash 450.

Each of the previously produced colouring films was folded centrally, parallel to its longitudinal axis. One hair strand in each case was wetted with water and then placed in the fold of a colouring film. The hair strand was then drizzled with the activator preparation. The colouring film was then in each case folded around the hair strand, such that the hair strand was fully encased by the chromatography paper disposed on the inner side of the colouring film and left there for about 30 minutes. The hair strand was then unwrapped again, washed with water and dried.

Each hair strand was then measured again colorimetrically.

The L-value indicates the brightness of the colouring (L=0=black; L=100=white). The ΔL value used for the assessment of the colour intensity is given from the L*a*b* colorimetric values as follows:

ΔL=Lᵢ−L₀

L₀: Colorimetric values before the colouring
Lᵢ: Colorimetric values after the colouring

The ΔL value indicates the difference in the brightness between the uncoloured and the coloured hair. The greater is the dL value, the greater is the difference in the brightness between the uncoloured and the coloured hair, and the more intense is the colouring on the hair.

The ΔE value used for the assessment of the colour intensity is given from the L*a*b* colorimetric values as follows:

ΔE=[(Lᵢ−L₀)²+(aᵢ−a₀)²+(bᵢ−b₀)]^{1/2}

L₀, a₀ and b₀: Colorimetric values before the colouring
Lᵢ, aᵢ and bᵢ: Colorimetric values after the colouring

The ΔE indicates the colour difference between the coloured and the treated hair strands. The greater is the dE value, the greater is the colour difference (i.e. the colour distance) between the uncoloured and the coloured strands, and the more intense is the colour.

### Kerling 9-0

| Colouring | | | | | | |
|---|---|---|---|---|---|---|
| film, coloured | Activator | | | | | |
| with colouring | preparation, | | | | | |
| solution | reaction time | L-value | a-value | b-value | ΔL-value | ΔE-value |
| untreated | — | 77.24 | 0.27 | 4.70 | — | — |
| FL1 | AZ1, 30 min | 49.52 | 9.62 | 17.99 | 27.72 | 31.74 |
| FL2 | AZ1, 30 min | 41.91 | 22.59 | 9.58 | 35.33 | 42.07 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for colouring human hair, comprising the following steps:
(I) bringing a hair strand into contact with a colouring film, wherein the colouring film comprises at least one absorbent material and at least one substantive dye,
(II) applying a water-containing activator preparation to the hair strand and/or the colouring film,
(III) encasing the hair strand with the colouring film,
(IV) leaving the activator preparation to take effect on the hair strand and the colouring film for a period of from about 1 to about 60 minutes, and
(V) removing the colouring film.

2. The method according to claim 1, comprising the step of
(I) bringing a hair strand into contact with a colouring film, wherein the colouring film comprises at least one cellulose-based absorbent material.

3. The method according to claim 1, comprising the step of
(I) bringing a hair strand into contact with a colouring film, wherein the colouring film comprises at least one absorbent material selected from the group of chromatography paper, filter paper, crepe paper, washi, China paper, and/or woven or non-woven cellulose fibres.

4. The method according to claim 1, comprising the step of
(I) bringing a hair strand into contact with a multi-layer colouring film, wherein an inner layer of the film, which is brought into contact with the hair strand, comprises an absorbent material.

5. The method according to claim 1, comprising the step of
(I) bringing a hair strand into contact with a multi-layer colouring film, wherein an outer layer of the film comprises a layer made of metal or a polymeric material.

6. The method according to claim 1, comprising the step of
(I) bringing a hair strand into contact with a colouring film, wherein the absorbent material is penetrated by at least one substantive dye.

7. The method according to claim 1, comprising the step of
(I) bringing a hair strand into contact with a colouring film, wherein the colouring film comprises at least one anionic dye.

8. The method according to claim 1, comprising the step of
(I) bringing a hair strand into contact with a colouring film, wherein the colouring film comprises at least one absorbent material and at least one substantive dye and at least one acid.

9. A method according to claim 1, wherein the activator preparation comprises at least one solvent selected from the group of benzyl alcohol, phenoxyethanol, propylene carbonate, ethylene carbonate, 1,2-propylene glycol, butylene carbonate, glyceryl carbonate, ethanol, ethylene glycol, isopropanol, 1,3-propylene glycol, glycerol, n-butanol and 1,3-butylene glycol.

10. The method according to claim 1, wherein the activator preparation comprises at least one acid selected from the group of lactic acid, citric acid, tartaric acid, malic acid, 1-hydroxyethane-1,1-diphosphonic acid, 2,6-dipicolinic acid, benzoic acid, phosphoric acid, sulfuric acid, hydrochloric acid, maleic acid, succinic acid, oxalic acid, ascorbic acid, phytic acid and/or gluconic acid.

11. The method according to claim 1, wherein the activator preparation has a pH value of from about 1.0 to about 7.0.

12. A cosmetic product, comprising
(A) at least one colouring film comprising at least one absorbent material and at least one substantive dye, and
(B) a water-containing activator preparation.

13. The method according to claim 2, comprising the step of
(I) bringing a hair strand into contact with a colouring film, wherein the colouring film comprises at least one absorbent material selected from the group of chromatography paper, filter paper, crepe paper, washi, China paper, and/or woven or non-woven cellulose fibres.

14. The method according to claim 1, comprising the step of
(I) bringing a hair strand into contact with a multi-layer colouring film, wherein an inner layer of the film, which is brought into contact with the hair strand, comprises an absorbent material selected from the group of chromatography paper, filter paper, crepe paper, washi, China paper, and/or woven or non-woven cellulose fibres.

15. The method according to claim 2, comprising the step of
(I) bringing a hair strand into contact with a multi-layer colouring film, wherein an inner layer of the film, which is brought into contact with the hair strand, comprises an absorbent material.

16. The method according to claim 3, comprising the step of
(I) bringing a hair strand into contact with a multi-layer colouring film, wherein an inner layer of the film, which is brought into contact with the hair strand, comprises an absorbent material.

17. The method according to claim 2, comprising the step of
(I) bringing a hair strand into contact with a multi-layer colouring film, wherein an inner layer of the film, which is brought into contact with the hair strand, comprises an absorbent material selected from the group of chromatography paper, filter paper, crepe paper, washi, China paper, and/or woven or non-woven cellulose fibres.

18. The method according to claim 3, comprising the step of
(I) bringing a hair strand into contact with a multi-layer colouring film, wherein an inner layer of the film, which is brought into contact with the hair strand, comprises an absorbent material selected from the group of chromatography paper, filter paper, crepe paper, washi, China paper, and/or woven or non-woven cellulose fibres.

19. The method according to claim 1, comprising the step of
(I) bringing a hair strand into contact with a colouring film, wherein the colouring film comprises at least one anionic dye selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 18, Acid Red 27, Acid Red 33, Acid Red 35, Acid Red 51, Acid Red 52, Acid Red 73, Acid Red 87, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 7, D&C Yellow 8, D&C Orange 4, D&C Green 5, D&C Orange 10, D&C Orange 11, D&C Red 21, D&C Red 27, D&C Red 33, D&C Violet 2 and/or D&C Brown 1.

20. The method according to claim 1, comprising the step of
(I) bringing a hair strand into contact with a colouring film comprising at least one absorbent material, at least one substantive dye, and at least one acid,
wherein an inner layer of the film, which is brought into contact with the hair strand, comprises the absorbent material selected from the group of chromatography paper, filter paper, crepe paper, washi, China paper, and/or woven or non-woven cellulose fibres,
wherein an outer layer of the film comprises a layer made of metal or a polymeric material,
wherein the absorbent material is penetrated by the at least one substantive dye,
wherein the colouring film comprises at least one anionic dye, which is selected from the group of Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 18, Acid Red 27, Acid Red 33, Acid Red 35, Acid Red 51, Acid Red 52, Acid Red 73, Acid Red 87, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 7, D&C Yellow 8, D&C Orange 4, D&C Green 5, D&C Orange 10, D&C Orange 11, D&C Red 21, D&C Red 27, D&C Red 33, D&C Violet 2 and/or D&C Brown 1,
wherein the activator preparation comprises at least one solvent selected from the group of benzyl alcohol, phenoxyethanol and propylene carbonate,
wherein the activator preparation comprises at least one acid selected from the group of lactic acid, citric acid, tartaric acid, malic acid, 1-hydroxyethane-1,1-diphosphonic acid, 2,6-dipicolinic acid, benzoic acid, phosphoric acid, sulfuric acid, hydrochloric acid, maleic acid, succinic acid, oxalic acid, ascorbic acid, phytic acid and/or gluconic acid, and
wherein the activator preparation has a pH value of from about 2.0 to about 3.5.
